(19) United States
(12) Patent Application Publication
(10) Pub. No.: US 2022/0050101 A1
(43) Pub. Date: Feb. 17, 2022

(54) DEVICE AND METHOD FOR MEASUREMENT OF SARS-CoV-2 SPECIFIC ANTIGEN IN A BIOLOGICAL SAMPLE

(71) Applicant: PATHSHODH HEALTHCARE PVT. LTD., Sanjaynagar (IN)

(72) Inventors: NAVAKANTA BHAT, Sanjaynagar (IN); VINAY KUMAR, Sanjaynagar (IN)

(73) Assignee: PATHSHODH HEALTHCARE PVT. LTD., Sanjaynagar (IN)

(21) Appl. No.: 17/322,392

(22) Filed: May 17, 2021

(30) Foreign Application Priority Data
May 15, 2020 (IN) 202041020469

(51) Int. Cl. G01N 33/543; G01N 1/38; G01N 33/569; B01L 3/00; B01L 9/00; G01N 27/327; G01N 27/48

(52) U.S. Cl. CPC G01N 33/54386; B01L 2300/126; G01N 33/56983; B01L 3/508; B01L 9/52; G01N 27/3277; G01N 27/3276; G01N 27/48; G01N 2333/165; G01N 2469/10; B01L 2200/025; B01L 2200/143; B01L 2300/023; B01L 2300/024; B01L 2300/027; B01L 2300/0645; B01L 2300/0663; B01L 2300/123; G01N 1/38

(57) ABSTRACT

A device for retaining a biological sample, for measuring a concentration of a SARS-CoV2 specific antigen, with SARS-CoV2 antigen-specific and electrochemically active immunoreceptor that is conjugated with an electrochemically active substance and optionally including an electrode reactivity enhancement agent and antibody stabilization agent. The immunoreceptor is configured to be in chemical contact with electrodes and a biological sample with SARS-CoV2 specific antigen of the device. The present invention also provides a device holder for holding the device of the present invention and a point-of-care biosensor. A method for measuring a concentration of SARS-CoV2 specific antigen from a reduced volume of biological sample is also provided in the presence of the antigen-specific and electrochemically active immunoreceptor, by measuring a peak value of redox current of the SARS-CoV2 antigen-specific and electrochemically active immunoreceptor and determining a concentration of SARS-CoV2 specific antigen in the biological sample, by linearly matching with a corresponding reference redox current.

### TECHNICAL FIELD

The subject matter of the present invention relates to a device, a device holder, a point-of-care biosensor for collecting and retaining a biological sample, for measuring a concentration of a SARS-CoV2 specific antigen in a biological sample. The present invention further provides a method for an accurate measurement of a concentration of SARS-CoV2 specific antigen, in a biological sample of reduced volume.

### BACKGROUND OF THE INVENTION

In December 2019, an outbreak of an unknown disease termed as pneumonia of unknown cause was reported in Wuhan, Hubei province, China. Since then the outbreak has spread rapidly to infect over thousands of people in China resulting in number of deaths. This outbreak also has spread to many other countries resulting in infections and deaths. The causative agent of this mysterious pneumonia was identified as a novel Coronavirus (nCoV) by several independent laboratories. The causative virus has been named as a severe acute respiratory syndrome Coronavirus 2 (SARS-CoV-2) and the corresponding\infection (disease) has been named as Coronavirus Disease 2019 (COVID-19) by the World Health Organization. According to the daily report of the World Health Organization, the epidemic of SARS-CoV-2, so far has registered 1,521,252 cases and 92,798 deaths worldwide by Apr. 10, 2020.

Coronaviruses (CoVs) are a group of highly diverse, enveloped, positive-sense, and single-stranded RNA viruses. They cause several diseases involving respiratory, enteric, hepatic and neurological systems with varying severity among humans and animals. Human Coronavirus (HCov) infections have traditionally caused a low percentage of annual respiratory infections, such as mild respiratory illness, resulting from HCoV-OC43, HCoV-229E, HCoVNL63 and HCoV-HKU1 human corona viruses. Over the past two decades, two novel Coronaviruses viz., severe acute respiratory syndrome CoV (SARS-CoV) and Middle East respiratory syndrome CoV (MERS-CoV), have emerged and caused severe human diseases. During the epidemic, SARS-CoV infected more than 8,000 people worldwide resulting in about 800 fatalities, representing its mortality rate of around 10%. Whereas MERS-CoV infected over 857 official cases and 334 deaths, making its mortality rate of about 35%.

SARS-CoV-2 is the seventh member of the family of CoVs that infects humans. The main symptoms of COVID-19 include fever, fatigue, and cough, which are similar to that of SARS-CoV and MERS-CoV infected subjects. There are some overlapping and discrete aspects of the pathology and pathogenesis of these CoVs which cause severe diseases in humans. The pathogen that causes COVID-19 is an nCoV that was first identified in January 2020 and named as SARS-CoV-2 (also known as 2019-nCoV).

The genome of CoV encodes four major structural proteins—spike (S), envelope (E), membrane (M), and nucleocapsid (N)—and approximately 16 nonstructural proteins (nsp1-16) and five to eight accessory proteins. Among them, the S protein plays an essential role in viral attachment, fusion, entry, and transmission. It comprises an N-terminal S1 subunit responsible for virus-receptor binding and a C-terminal S2 subunit responsible for virus-cell membrane fusion. S1 is further divided into an N-terminal domain (NTD) and a receptor-binding domain (RBD). SARS-CoV-2 and SARS-CoV bind angiotensin converting enzyme 2 (ACE2). Phylogenetically, SARS-CoV-2 is closely related to SARS-CoV, sharing approximately 79.6% genomic sequence identity. During infection, CoV first binds the host cell through interaction between its S1-RBD and the cell membrane receptor, triggering conformational changes in the S2 subunit that result in virus fusion and entry into the target cell.

Since, the start of the COVID-19 pandemic, the World Health Organization (WHO) has emphasized the crucial importance of testing. Testing is the basis of public health detective work to shut down an epidemic. There are two types of tests that laboratories carry out for COVID-19. The first one is to confirm if the body currently has the virus, which is done through PCR test to measure the virus genetic material. The second type of test is to detect if a patient's body has made antibodies to fight against the virus, which is commonly called the antibody test. The PCR test detects the virus and it is important to determine if someone who is very ill has COVID-19. The test uses swabs from the nose and throat and has a high accuracy rate. It's worth noting that PCR tests can be very labour intensive with several stages at which errors may occur between sampling and analysis. Per sample testing time in PCR is high and that is a limitation at the time of pandemic outburst.

COVID-19 antigen testing plays a vital role to take the definite decision about the infection status of the patient and has been the gold standard in COVID-19 diagnosis. Commercially available COVID-19 tests currently fall into two major categories. The first category includes molecular assays for detection of SARS-CoV-2 viral RNA using polymerase chain reaction (PCR)-based techniques or nucleic acid hybridization-related strategies. The second category includes serological and immunological assays that largely rely on detecting antibodies produced by individuals as a result of exposure to the virus or on detection of antigenic proteins in infected individuals. It is important to reemphasize that these two categories of tests serve overlapping purposes in management of the COVID-19 pandemic. Testing for SARS-CoV-2 Antigen in infected individuals during the acute phase of infection is very crucial for healthcare professionals to decide the next step.

### OBJECTS OF THE PRESENT INVENTION

The present invention provides a device for collecting and retaining a biological sample and to measure a concentration of a SARS-CoV2 specific antigen, through a SARS-CoV2 antigen-specific and electrochemically active immunoreceptor that is conjugated with at least an electrochemically active substance. The concentration of SARS-CoV2 antigen is measured electrochemically, by contacting SARS-CoV2 antigen-specific and electrochemically active immunoreceptor with an electrode arrangement of the device and the biological sample.

An object of the present invention is to provide a device with the SARS-CoV2 antigen-specific and electrochemically active immunoreceptor along with an electrode reactivity enhancement agent.

Another object of the present invention is to provide a device with the SARS-CoV2 antigen-specific and electrochemically active immunoreceptor, along with an electrode reactivity enhancement agent and an antibody stabilization agent.

Yet another object of the present invention is to provide a device holder for holding the device with the SARS-CoV2 antigen-specific and electrochemically active immunoreceptor.

It is also an object of the present invention to provide a point-of-care device or a biosensor with the SARS-CoV2 antigen-specific and electrochemically active immunoreceptor, for measuring a concentration of a SARS-CoV2 specific antigen, in a biological sample of reduced volume, through a measurement of redox current, on an application of a redox potential.

Yet another object of the present invention is to provide a method for a qualitative and quantitative measurement of concentration of a SARS-CoV2 specific antigen, in a reduced amount of a biological sample, using the device with the SARS-CoV2 antigen-specific and electrochemically active immunoreceptor.

### SUMMARY OF THE PRESENT INVENTION

The present invention provides a device for collecting and retaining a biological sample, for measuring a concentration of a SARS-CoV2 specific antigen with SARS-CoV2 antigen-specific and electrochemically active immunoreceptor that is conjugated with at least an electrochemically active substance, is configured to be in chemical contact with electrodes and a biological sample with SARS-CoV2 specific antigen. The present invention also provides a device holder for holding the device of the present invention. The present invention also provides a point-of-care biosensor with the device of the present invention, for measuring a concentration of a SARS-CoV2 specific antigen in a biological sample, including a processing means to measure a peak value of redox current of the SARS-CoV2 antigen-specific and electrochemically active immunoreceptor, from a redox potential applied to the device, to measure a concentration of SARS-CoV2 specific antigen in the biological sample, by linearly matching the measured redox current with a corresponding reference redox current of the device and retrieving the matched concentration of the SARS-CoV2 specific antigen for display. A method for measuring a concentration of SARS-CoV2 specific antigen, from a reduced volume of biological sample is also provided, comprising the steps of collecting a desired biological sample of reduced volume, contacting the biological sample with the SARS-CoV2 antigen-specific and electrochemically active immunoreceptor of the device, measuring a peak value of redox current of the SARS-CoV2 antigen-specific and electrochemically active immunoreceptor of the device and determining a concentration of SARS-CoV2 specific antigen in the biological sample, by linearly matching the measured redox current with a corresponding reference redox current of the device and retrieving the matched concentration of the SARS-CoV2 specific antigen, and displaying the concentration of the SARS-CoV2 specific antigen.

### BRIEF DESCRIPTION OF THE DRAWINGS

FIG. 1 is a schematic exploded view of a device with a two-electrode arrangement, along with a SARS-CoV2 antigen-specific and electrochemically active immunoreceptor with electrode reactivity enhancement agent and an antibody stabilization agent, for collecting and retaining a biological sample, to detect and measure the concentration of a SARS-CoV2 specific antigen.

FIG. 2 is a schematic exploded view of a device with a three-electrode arrangement, along with a SARS-CoV2 antigen-specific and electrochemically active immunoreceptor with electrode reactivity enhancement agent and an antibody stabilization agent, for collecting, retaining a biological sample, to detect and measure the concentration of a SARS-CoV2 specific antigen.

FIG. 3 is a schematic exploded view of a device with two pairs of three-electrode arrangement and trays, along with a SARS-CoV2 antigen-specific and electrochemically active immunoreceptor with electrode reactivity enhancement agent and an antibody stabilization agent, to detect and measure the concentration of SARS-CoV-2 specific antigen.

FIG. 4(A) is a schematic top view of a device with a three-electrode arrangement, along with a SARS-CoV2 antigen-specific and electrochemically active immunoreceptor with electrode reactivity enhancement agent and an antibody stabilization agent, to detect and measure the concentration of SARS-CoV-2 specific antigen, in different wells.

FIG. 4(B) is a schematic cross-sectional view of a device in which the SARS-CoV2 antigen-specific and electrochemically active immunoreceptor with electrode reactivity enhancement agent and an antibody stabilization agent is arranged on the surface of a receptor-membrane.

FIG. 4(C) is a cross-sectional view of a device, in which the SARS-CoV2 antigen-specific and electrochemically active immunoreceptor with electrode reactivity enhancement agent and an antibody stabilization agent is arranged on the surface of the electrodes.

FIG. 4(D) is a cross-sectional view of a device, in which the SARS-CoV2 antigen-specific and electrochemically active immunoreceptor with electrode reactivity enhancement agent and an antibody stabilization agent is embedded along with the electrode.

FIG. 4(E) and FIG. 4(F) are cross-sectional views of a device, in which the SARS-CoV2 antigen-specific and electrochemically active immunoreceptor with electrode reactivity enhancement agent and an antibody stabilization agent is in a liquid phase and disposed on the electrode with membrane (FIG. 4(E)) or without a membrane (FIG. 4(F)), during a testing period.

FIG. 5 is a perspective view of an exemplary device holder for the device of the present invention.

FIG. 6(a) is a perspective view of an exemplary point-of-care biosensor, holding the device of the present invention, for measuring a concentration of a COVID-19 specific antigen, in a biological sample.

FIG. 6(b) is a schematic depiction of broad internal electronic architecture of the point-of-care biosensor.

FIG. 7 depicts the steps of the method of detection and measurement of SARS-CoV2 specific antigen, in a biological sample.

FIG. 8(a) illustrates the steps in the preparation of an exemplary SARS-CoV2 antigen-specific and electrochemically active immunoreceptor and biological sample.

FIG. 8(b) illustrates a reaction mechanism of antigen-antibody binding, in accordance with the method of the present invention.

FIG. 9(A) and FIG. 9(B) depict a cyclic voltammetry detection technique of the present invention, to measure the SARS-CoV2 specific antigen, where SARS-CoV2 antibody that is tagged with an electrochemically active label horseradish peroxidase (HRP), binds with SARS-CoV2 specific antigen and provides a changed redox signal.

FIG. 10(A)-(F) illustrate different electrochemical techniques, which can be adapted for use for the electrochemical detection of SARS-CoV-2 antigen, in accordance with an embodiment of the present invention.

FIG. 11 is a graphical illustration of detection and measurement of SARS-CoV-2 antigen, in the absence of an electrode reactivity enhancement agent.

FIG. 12 is a graphical illustration of detection and measurement of SARS-CoV-2 antigen, in the presence of an electrode reactivity enhancement agent.

FIG. 13 is a graphical illustration of quantitative estimation of SARS-CoV-2 antigen, in the absence of an electrode reactivity enhancement agent.

### DETAILED DESCRIPTION OF THE INVENTION

Accordingly, the present invention provides a device with a SARS-CoV2 antigen-specific and electrochemically active immunoreceptor with an electrode reactivity enhancement agent and an antibody stabilization agent for collecting and retaining biological samples. The present invention also provides for a device holder for holding the device. A point-of-care biosensor with the SARS-CoV2 antigen-specific and electrochemically active immunoreceptor, is also provided for measuring a concentration of a COVID-19 specific antigen in a biological sample. The present invention further provides a method for an electrochemical detection and measurement of concentration of a SARS-CoV2 specific antigen, in biological samples of reduce volume.

The SARS-CoV2 antigen-specific and electrochemically active immunoreceptor of the device of the present invention binds with the spike protein (S protein) that is present on the surface of SARS-CoV-2 virus. Since the immunoreceptor is specific to spike protein (S protein), the subject matter of the present invention can be used to detect different variants of SARS-CoV2 virus, as long as the spike protein is conserved.

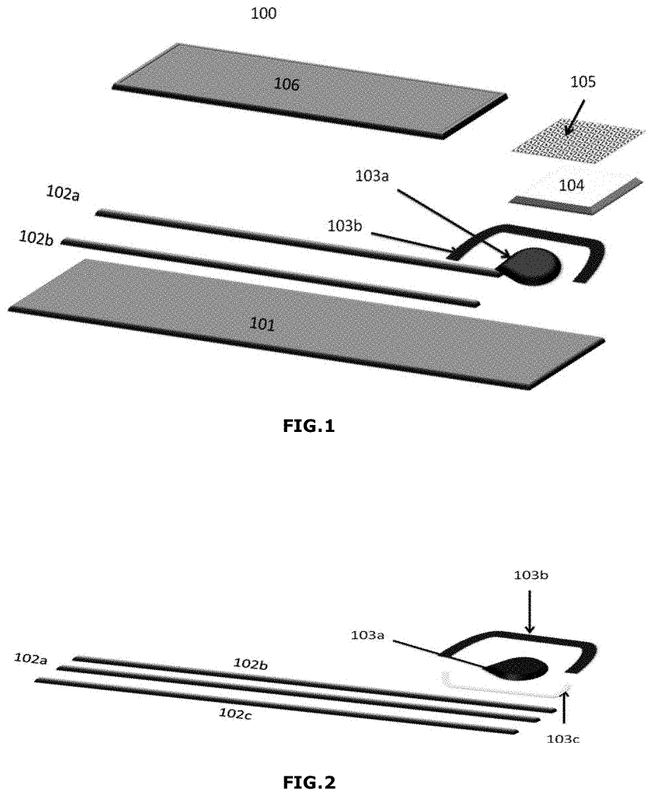

Now, the preferred embodiments of the device for collecting and retaining a biological sample, for measuring a concentration of a SARS-CoV2 specific antigen in a biological sample, are described by initially referring to FIG. 1 of the accompanied drawings.

FIG. 1 depicts the electrochemically active device that is adapted to collect and retain a biological sample, for subsequent measurement of SARS-CoV2 specific antigen, in a selected biological sample. The device 100 as shown in FIG. 1 is provided with a substrate 101, which acts as base on which other constituents of the device 100 are arranged. In this embodiment, the substrate 101 is exemplarily shown as an elongated rectangular structure. However, it is understood here that the substrate 101 can take other shapes such as square, circular depending on the shape and configuration of a device holder or a biosensor that is used in conjunction with the device 100. The substrate 101 can be made of any suitable rigid or flexible material that is suitable for the incorporation of patterned electrodes. For instance, materials such as polyvinylchloride (PVC), polyethylene terephthalate (PET), polymethylmethacrylate (PMMA), epoxy fiber composites, polyamides composites and paper can be used as preferred materials for the substrate 101. Whereas, the preferred rigid materials for the substrate 101 can be ceramic, glass or any other like materials. In any case, the selection of suitable material for the substrate 101 is made to ensure that the substrate 101 can not only provide a desirable strength and flexibility but also can act as an electrical insulator. Advantageously, the substrate 101 is hydrophilic in nature to prevent percolation of the biological sample, when it comes in physical contact with the substrate 101. The edges of the substrate 101 are also provided with suitable profiles, such as tapered or curved, to facilitate an easy ingress into and egress out of the selected device holder or the biosensor.

A pair of conductive tracks 102a and 102b are arranged on the substrate 101. The conductive tracks 102a and 102b are formed by using any patterning method such as screen printing, lithography, thermal evaporation, sputtering, laser patterning, preferably screen-printing. In an exemplary aspect, in FIG. 1, a pair of conductive tracks 102a and 102b are formed for implementation. However, the required number of conductive tracks can be suitably increased or varied. The routing of the conductive tracks 102a and 102b are exemplarily shown as straight tracks in FIG. 1. Other suitable configurations for the conducting tracks such as polygons can be used. The material for the conductive tracks 102a and 102b can be an electrically conductive material, such as copper, aluminium, gold, silver, platinum, mercury, carbon, glassy carbon and graphite or any other suitable electrically conducting materials or alloys of these materials. The conducting tracks 102a and 102b are used to establish an electrical connection with the device, device holder and point-of-care biosensor of the present invention as hereinafter described.

Pair of electrodes 103a and 103b are electrically connected to the conducting tracks 102a and 102b respectively, as shown in FIG. 1. The electrodes 103a and 103b are overlaid on the conducting tracks 102a and 102b and arranged at the terminal ends of the conducting tracks 102a and 102b, so as to form a layer above the conducting tracks 102a and 102b, as shown in FIG. 1. The material for the electrodes 103a and 103b is selected from metals or alloys, which are electrochemically active, such as gold, platinum, mercury, carbon, glassy carbon and graphite. In the exemplary arrangement of electrodes as shown in FIG. 1, the electrode 103a acts as a working electrode and whereas the electrode 103b takes up the role of a counter electrode/a reference electrode.

A membrane 104 is arranged on the pair of electrodes 103a and 103b as shown in FIG. 1, which acts as a base member. The membrane 104 can be a blank membrane or can act as an integration membrane for a SARS-CoV2 antigen-specific and electrochemically active immunoreceptor 105.

The electrochemically active immunoreceptor 105 is optionally provided with an electrode reactivity enhancement agent and an antibody stabilization agent as hereinafter described. The material for the membrane 104 can be polymer, cellulose, nitrocellulose, nylon, cotton fabric, filter paper or any other commercially available membranes such as BIODYNE membrane from PALL life-sciences and GE Healthcare membranes. Accordingly, the SARS-CoV2 antigen-specific and electrochemically active immunoreceptor, along with optional electrode reactivity enhancement agent and the stabilization agent is in chemical contact with the membrane 104.

The SARS-CoV2 antigen-specific and electrochemically active immunoreceptor 105 that is configured to be in chemical contact with the at least two-electrode member includes antibodies such as monoclonal and polyclonal antibodies that are specific to SARS-CoV2 antigen. A combination of these antibodies can also be suitably used. In the present invention, preferably antibody of the type human immunoglobulin M(IgM) antibody or human immunoglobulin G(IgG), or a combination thereof, is used as the SARS-CoV2 antigen-specific and electrochemically active immunoreceptor 105.

The antibodies that are used as SARS-CoV2 antigen-specific and electrochemically active immunoreceptor 105 can be selected from the ones that are expressed in a host cell of any species such as E. coli, human cells or mammalian cells.

In another aspect of the present invention, the SARS-CoV2 antigen-specific and electrochemically active immunoreceptor 105, is tagged or conjugated with an electrochemically active substance to impart redox activity. The electrochemically active substance is preferably selected from substances such as horseradish peroxidase (HRP), histidine, biotin, alkaline phosphatase or a combination of these substances. It is also within purview of this invention to use metals or alloys of metals such as gold and silver, as the electrochemically active substance.

In another aspect of the present invention, the SARS-CoV2 antigen-specific and electrochemically active immunoreceptor 105 includes at least an electrode reactivity enhancement agent. In the present embodiment the preferred electrode reactivity enhancement agent is selected from materials such as reduced graphene oxide (rGO), carbon nanotubes (CNT), metal nano particles, such as gold and silver), metal oxide nano particles, such as zinc oxide, cobalt oxide) or a combination of these materials. These electrode reactivity enhancement agents enhance the reactivity of carbon electrodes and thereby enhance the redox currents, which is particularly significant while detecting extremely low concentrations of SARS-CoV2 antigen.

In yet another aspect of the present invention, the SARS-CoV2 antigen-specific and electrochemically active immunoreceptor 105 includes at least an antibody stabilization agent, such as ELISA stabilization buffer, a plate stabilizer or a combination of these agents. These agents help stabilize the immunoreceptor on electrodes so that there is no degradation, which is important for storing the device for a long term in order to extend its shelf life.

In accordance with another aspect of the invention a cartridge or a cassette is adapted for housing the device 100 of the present invention.

Now, the preferred embodiments preparation of the SARS-CoV2 antigen-specific and electrochemically active immunoreceptor 105 and its initiation of chemical contact with the at least pair of electrodes 103a, 103b are described.

A solution of the SARS-CoV2 antigen-specific and electrochemically active immunoreceptor 105 is prepared and dispensed on at least the pair of electrodes 103a, 103b and/or the membrane 104 and dried to form a solid chemical layer on at least the pair of electrodes 103a, 103b and/or the membrane 104.

A passivation layer 106 is arranged to cover at least the pair of conductive tracks 102a, 102b, as shown in FIG. 1. The passivation layer 106 is used to provide protection for the conductive elements of the device 100 and to precisely define the electrode region.

In an alternate embodiment, a solution of the SARS-CoV2 antigen-specific and electrochemically active immunoreceptor 105 is pre-mixed with a selected biological sample and a reduced volume of the pre-mixed solution is dispensed on the at least the pair of electrodes 103a, 103b and/or membrane 104 during the testing for the presence of SARS-CoV2 specific antigen.

In yet another aspect of the present invention, as shown in FIG. 2, an arrangement of set of three electrodes 103a, 103b and 103c is implemented in conjunction with SARS-CoV2 antigen-specific and electrochemically active immunoreceptor 105 (as shown in FIG. 1), where the electrodes 103a, 103b and 103c are connected to the conducting tracks 102a, 102b and 102c respectively, to collect and retain a selected biological sample. The increased number of electrodes facilitates the detection of a single bio-analyte (SARS-CoV2 specific antigen) in the biological sample with an increased accuracy, since no current flows through reference electrode. In this implementation the electrode 103c acts as a reference electrode. The preferred material for the reference electrode 103c is silver (Ag), a silver chloride (AgCl), silver/silver chloride (Ag/AgCl), carbon or saturated calomel, which gives a stable reference potential.

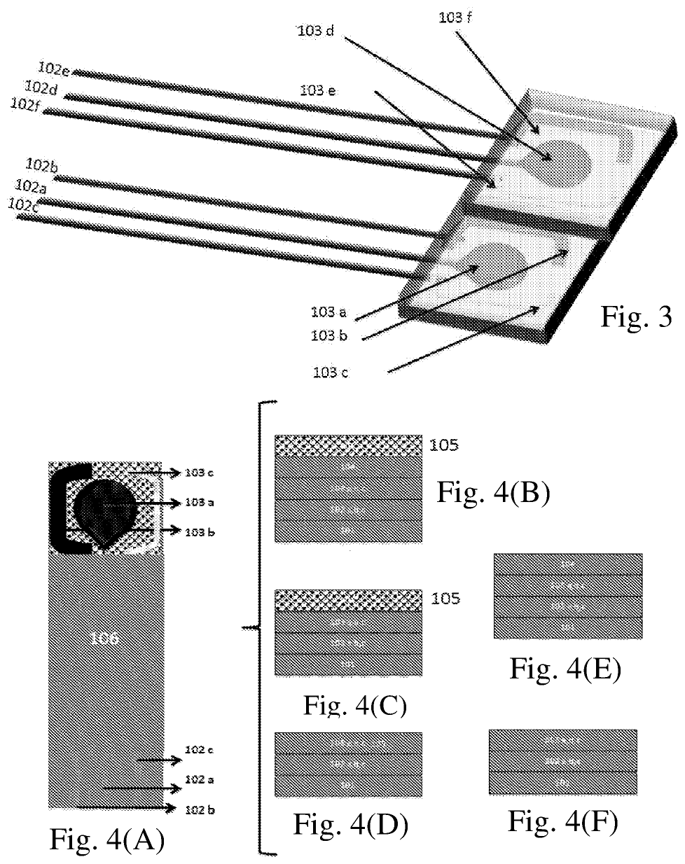

In yet another aspect of the present invention, two pairs or sets of three-electrodes 103a, 103b, 103c, 103d, 103e and 103f are arranged on the conducting tracks 102a, 102b, 102c, 102d, 102e and 102f and are adapted for use to measure the multiple antigens on same electrode, in separate areas (wells), as shown in FIG. 3.

As shown in FIG. 4(A), depicts a top view of the device 100 with SARS-CoV2 antigen-specific and electrochemically active immunoreceptor 105 for the detection of SARS-CoV2 antigen, in a three-electrode arrangement 102a, 102b and 102c. FIG. 4(B), which is a corresponding cross-sectional view, depicting a substrate 101 on the surface of which conducting tracks 102a, 102b, 102c are arranged. A 3-electrode arrangement with a working electrode 103a, counter electrode 103b and reference electrode 103c that are connected to the conducting tracks 102a, 102b, 102c. The membrane 104 is arranged on the surface of the electrodes 103a, 103b and 103c. The receptor layer (SARS-CoV2 antigen-specific and electrochemically active immunoreceptor 105) with the optional electrode reactivity enhancement agent and antibody stabilization agent, is arranged on the surface of the membrane 104.

FIG. 4(C), which is a corresponding cross-sectional view depicting a substrate 101 on the surface of which conducting tracks 102a, 102b, 102c are arranged. A 3-electrode arrangement with a working electrode 103a, counter electrode 103b and reference electrode 103c connected to the conducting tracks 102a, 102b, 102c. The receptor (SARS-CoV2 antigen-specific and electrochemically active immunoreceptor 105) with the optional electrode reactivity enhancement agent and antibody stabilization agent, is arranged on surface of the electrodes 103a, 103b and 103c.

FIG. 4(D), which is a corresponding cross-sectional view depicting a substrate 101 on the surface of which conducting tracks 102a, 102b, 102c are arranged. A 3-electrode arrangement with a working electrode 103a, counter electrode 103b and reference electrode 103c connected to the conducting tracks 102a, 102b, 102c, where the electrodes are treated with the receptor 105 (SARS-CoV2 antigen-specific and electrochemically active immunoreceptor 105). The receptor (SARS-CoV2 antigen-specific and electrochemically active immunoreceptor 105) is optionally provided with the electrode reactivity enhancement agent and antibody stabilization agent.

FIGS. 4(E, F), which are corresponding cross-sectional views of the device 100, depicts a substrate 101 on the surface of which the conducting tracks 102a, 102b, 102c are arranged. A 3-electrode arrangement with a working electrode 103a, counter electrode 103b and reference electrode 103c are connected to the conducting tracks 102a, 102b, 102c. The electrodes 103a, 103b and 103c are either covered by membrane 104(4e) or left open (4f). The receptor 105 is prepared separately in liquid phase and premixed with biological sample and then applied at the time of testing. The receptor (SARS-CoV2 antigen-specific and electrochemically active immunoreceptor 105) is optionally provided with the optional electrode reactivity enhancement agent and antibody stabilization agent. The preferred embodiments as shown in FIGS. 4(A), (B), (C), (D), (E) and (F) are used to measure SARS-CoV2 antigen in a selected biological sample.

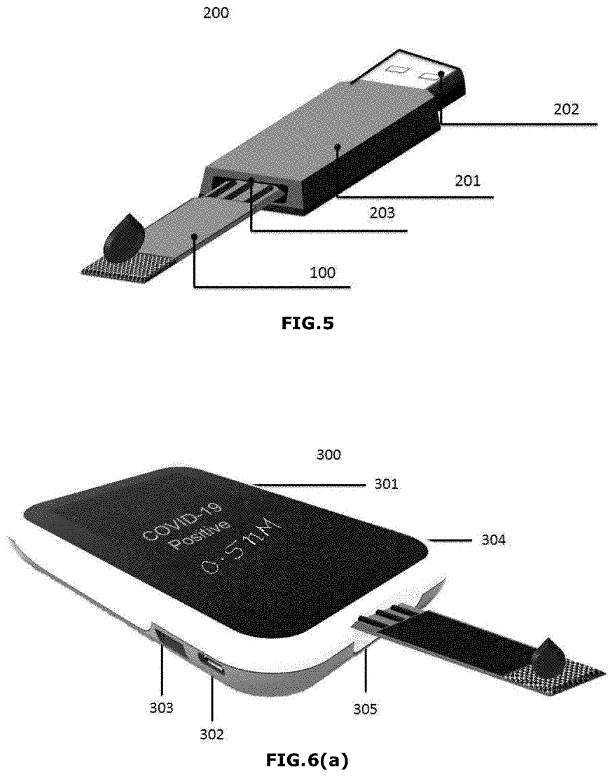

The preferred embodiments of the device holder 200 are now described by referring to FIGS. 4(A)-(F). The device holder 200 comprises a housing 201 with device detection and internal circuitry and the housing 201 that is adapted to connect to a processor (a digital micro processor) and a display member (not shown in this Figure). A device insertion port 203 is provided in the housing 201. The device 100, which is permitted to pass through the device insertion port 203, includes the substrate with at least the two-electrode member along with SARS-CoV2 antigen-specific and electrochemically active immunoreceptor, that is configured to receive a bio sample 204. A USB plug 202 is connected to the housing 201 as shown in FIG. 5. The device holder 200 is used to collect and retain the biological sample for subsequent testing. The device holder 200 is also provided with device detection, signal conditioning and data acquisition features to identify the type of bioanalyte (SARS-CoV2 antigen) that is stored on the device 100. The device holder 200 enables a user to insert the holder 200 into a biosensor (as hereinafter described) and collect the biological sample for a subsequent measurement of the bioanalyte.

The functional aspects of the device holder 200 are now described for measuring the concentration of SARS-CoV2 antigen in a selected biological sample. The device holder 200 is powered ON after connecting it to a processing and a display unit. The device 100 is then loaded into the device holder 200. The device detection circuitry (electronic circuitry) inside the housing 200 is adapted to indicate the detection of the designated device. When the device holder 200 detects the device 100, the device 100 is loaded with a selected biological sample and a desired redox potential is applied by the internal circuitry through a digital-to-analog converter (DAC) to the working electrode of the device 100 with respect to the reference electrode. The redox signal is measured by internal circuitry.

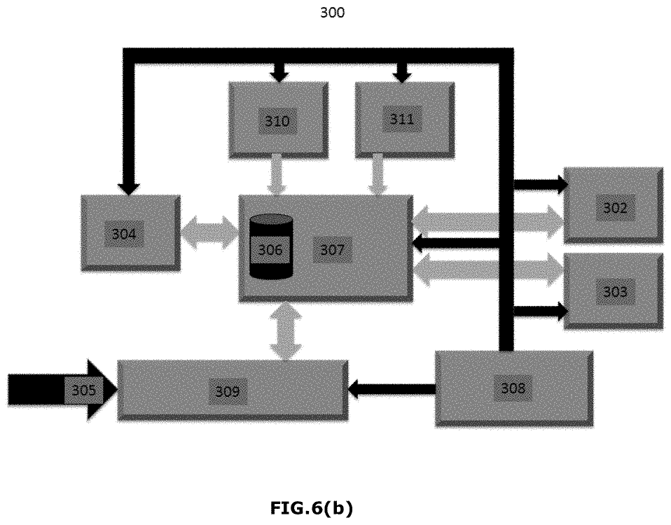

The point-of-care biosensor 300 for sensing a bioanalyte (SARS-CoV2 antigen) in a biological sample, as shown in FIG. 6(a). The point-of-care biosensor 300 comprises a housing 301. A micro USB 302 and micro SD card 303, are adapted to connect to the housing 300, through respective ports. The micro USB 302 is advantageously used to charge the biosensor 300 and micro SD card is used as a storage device. The housing 301 is also provided with display member 304, which can be an LCD, LED, OLED, OMLED, TFT or any other such display devices, including touch-sensitive devices to display an output of the point-of-care biosensor 300. A device insertion port 305 is provided in the housing 301. The device insertion port 305 is provided with a metallic contact to engage electrically with the device 100. In other words, the insertion port 305 is provided to receive the device 100, through the electrode members of the device 100. The point-of-care biosensor 300 is configured to facilitate a user to use the device 100, in a simple way, along with the point-of-care biosensor 300. The device 100 is initially inserted into the loaded point-of-care biosensor 300 and loaded with a selected biological sample of reduced volume, in the range of 1-150 μL, which entails a minimum invasive means in collecting the biological sample. The user is also at liberty to use the biosensor 300 at a room temperature and without concerning about other environmental factors such as humidity, temperature variation and storage conditions. The user by using the biosensor 300 is able to measure the concentration levels of the desired bioanalyte (SARS-CoV2 antigen), in a substantially shorter period of time. The user is provided with an instantaneous and accurate display of the concentration of the bioanalyte (SARS-CoV2 antigen) on the display member 304, since the inherent binding nature of bioanalyte (SARS-CoV2 antigen) is used in the biosensor 300 to measure the concentration levels.

Now, referring to FIG. 6(b), an internal electronic hardware architecture of the biosensor 300 is described. A database member 306 is provided in the housing 301 to store standard values of redox signal values such as voltage sweep rate, pulse amplitude and duration, redox potential, redox current and concentrations of the bioanalyte (SARS-CoV2 antigen) in the biological samples. The database 306 also incorporates the data pertaining to historical and current data of type and concentrations of the bio-analytes. The executables that are required to perform the various functions of the biosensor 300 are stored on a medium of the biosensor 300. A processing means such as a digital controller or a digital processor 307 is provided in the housing 301 and connected to the database member 306 and configured to apply appropriate electrochemical excitation to at least a two-electrode member having with electrochemically active immunoreceptor and measure corresponding electrochemical response. The digital controller 307 is arranged to measure a redox signal in the sample by linearly matching with the value of type and concentration of bio-analyte and display the qualitative or quantitative or both result of SARS-CoV2 antigen.

Power supply to the biosensor 300 is regulated by a power supply unit 308, which is connected to the biosensor 300. The power supply unit 308 includes both online and offline rechargeable battery with charging circuitry. A signal conditioning and device detection unit 309 is connected to the microcontroller or a digital processor 307 to detect the presence of the device 100 in the biosensor 300 and to apply appropriate electrochemical excitation and measuring the corresponding electrochemical response from the selected biological sample. Humidity and temperature sensors 310 and 311 are arranged in the housing 301. Once the measurement of the concentration levels of the bioanalyte (SARS-CoV2 antigen) is completed by the microcontroller 307, the concentration levels are displayed on the display member 304, along with historical data of the concentration levels of the bioanalyte (SARS-CoV2 antigen).

The point-of-care biosensor 300 of the present invention is configured to generate the redox signals, which can be an electrochemical signal, but not limited to a charge, current, potential or an impedance. In the present disclosure the use a redox current signal for the detection of SARS-CoV2 antigen is illustrated.

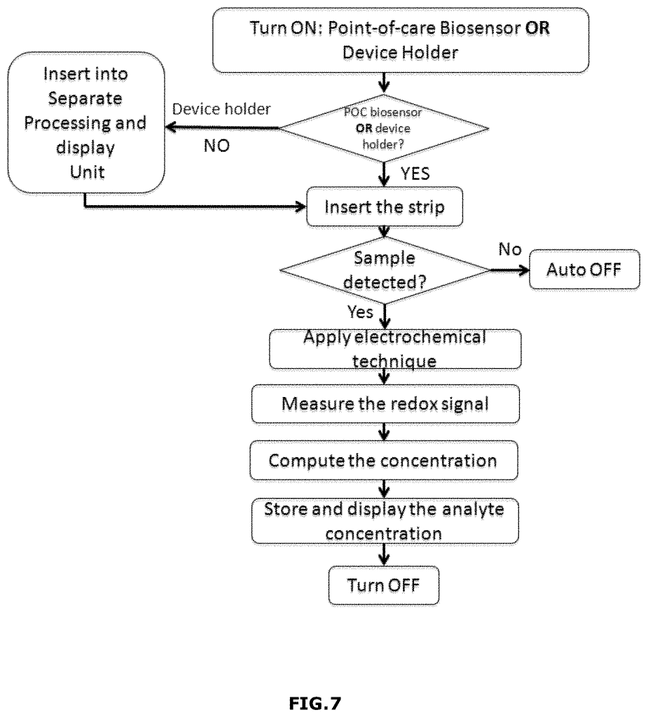

The present invention also provides a method for an accurate detection and measurement of concentration SARS-CoV2 antigen, both quantitatively and qualitatively, in a selected biological sample. The preferred embodiments of the method are now described by referring to FIG. 7.

In the method of the present invention, the determination and accurate measurement of SARS-CoV2 antigen in a biological sample, is performed by implementing the principle of electrochemistry.

The preferred biological samples include urine, blood, saliva, sweat, serum, or a nasopharyngeal culture, which are prepared or diluted in any suitable media such as saline buffer or stored in a suitable viral transport media (VTM) etc., are collected in small or reduced volumes, which are preferably in micro litres (μL). In the method of present invention, the preferred volume of the biological sample that can be used for the measurement of bioanalyte (SARS-CoV2 antigen) is in the range of 1-50 microlitres (μL) and the saline buffer volume is in the range of 10-100 microlitres (μL). The required volume of the biological sample is subject to the size of the surface area of the SARS-CoV2 antigen-specific and electrochemically active immunoreceptor of the device. The reduced collection of sample substantially reduces trauma in the subjects, since it is obtained through a minimally invasive sample extraction technique. The reduced volume of biological samples avoids the need for phlebotomy collection products.

In the method of present invention, the SARS-CoV2 antigen-specific and electrochemically active immunoreceptor substance is selected from a list of SARS-CoV2 specific antibodies, that are conjugated with at least an electrochemically active substance. Accordingly, antibodies thus selected are monoclonal antibodies or polyclonal antibodies, preferably human immunoglobulin M(IgM) or human immunoglobulin G(IgG), or a combination of these antibodies.

The selected SARS-CoV2 antigen-specific and electrochemically active immunoreceptor substance is advantageously tagged or conjugated with at least an electrochemically active substance, to form an antibody-electrochemically active substance conjugate.

In this method, the preferred electrochemically active substance is selected from enzymes such as horseradish peroxidase (HRP), alkaline phosphatase (ALP) and amino acid such as histidine, biotin (B7). A combination of these electrochemically active substances can also be suitable adapted for use. In addition, the electrochemically active substance is also selected from metals such as gold (Au) and silver (Ag) or an alloy of these metals.

The conjugated SARS-CoV2 antigen-specific and electrochemically active immunoreceptor also comprises or treated with at least an electrode reactivity enhancement agent, selected from reduced graphene oxide (rGO), carbon nanotubes (CNT), metal nano particles, such as, gold, silver, metal oxide nano particles, such as, zinco oxide and cobalt oxide. It is also within purview of this invention to use a combination of these electrode reactivity enhancement agents. These agents enhance the reactivity of carbon electrodes and thereby enhance the redox currents, which is especially important while detecting extremely low concentration of SARS-CoV2 antigen.

In yet another aspect of the present invention, the SARS-CoV2 antigen-specific and electrochemically active immunoreceptor includes at least a stabilization agent, selected from ELISA stabilization buffer, a plate stabilizer or a combination thereof. These agents help stabilize the immunoreceptors on electrodes so that there is no degradation, which is crucial for storing the device for long term to extend the shelf life.

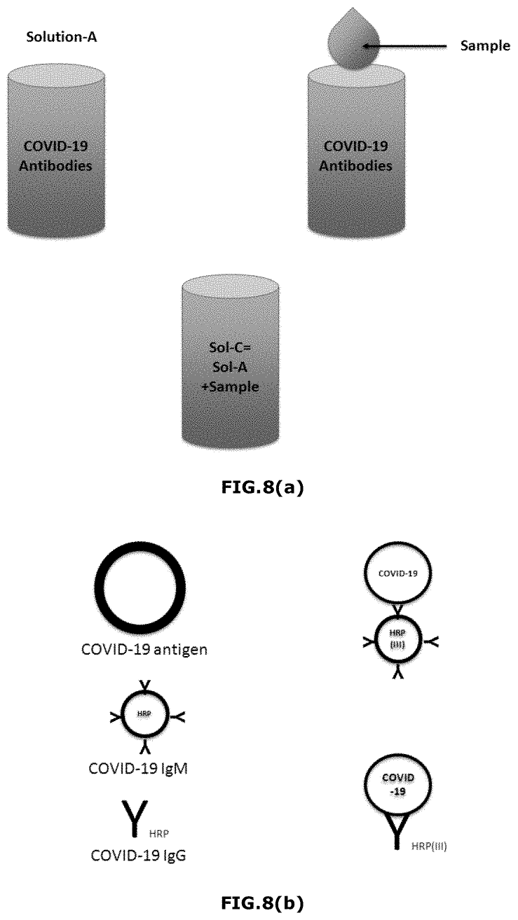

In a preferred embodiment of the method of the present invention, the SARS-CoV2 antigen-specific and electrochemically active immunoreceptor substance is prepared, advantageously as a solution of preferred chemical substances, as shown in FIG. 8(a). It is also understood here that the SARS-CoV2 antigen-specific and electrochemically active immunoreceptor substance can also be suitably prepared by using saline buffer or phosphate buffer solution.

For instance, in case HRP tagged/conjugated SARS-CoV2 specific antibody is selected as a preferred immunoreceptor, HRP tagged/conjugated SARS-CoV2 antibody is dissolved, by using ELISA techniques or can be prepared separately in another aqueous solution or any other solvents such as saline buffer or phosphate buffer solution, which can dissolve these substances. Subsequent to the preparation of the immunoreceptor substance, the selected electrode reactivity enhancement agent and an antibody stabilization agent is mixed as required.

The solution of the SARS-CoV2 antigen-specific and electrochemically active immunoreceptor substance thus prepared, is applied to the electrode members or membranes of the device of the present invention, as the case may be, to form a dry chemical layer of immunoreceptor, prior to the application of biological samples, so that the immunoreceptor is in chemical contact with the electrodes.

Alternately, the electrochemically active immunoreceptor solution can also be premixed with the biological samples and the mixed solution is applied to or contacted with the electrode members or membranes of the device.

The electrochemically active immunoreceptor solution can also be first applied to or contacted with the electrode and subsequently the selected biological sample is applied to or contacted with the electrode members or membranes of the device.

Alternatively, the desired biological sample can be first applied to the electrode and thereafter the drops of SARS-CoV2 antigen-specific and electrochemically active immunoreceptor substance are applied to the electrode members or to the membrane of the device.

The electrochemically active immunoreceptor solution with an electrode reactivity enhancement agent and an antibody stabilization agent can also be first applied at the electrode and dried at the electrode surface before biological samples is applied to the electrode members or membranes of the device.

It is also within the purview of the method of the present invention, where the biological samples that are applied to the electrode members or membranes of the device can be diluted in suitable solvent such as saline or phosphate buffer solution or stored in viral transport media (VTM).

Once the electrodes of the device with the SARS-CoV2 antigen-specific and electrochemically active immunoreceptor is ready, a biological sample of reduced volume, in which the concentration of the SARS-CoV2 antigen is to be detected and measured, is brought in chemical contact with the immunoreceptor of the device.

Thereafter, the antigen-antibody binding reaction is permitted to be stabilized over few minutes, preferably in the range of 1 to 10 minutes.

The reaction mechanism of the antigen-antibody binding, in accordance with the method of the present invention, is as illustrated in FIG. 8(b). The antibody tagged with HRP binds at the specific binding region of the antigen, such spike protein S1.

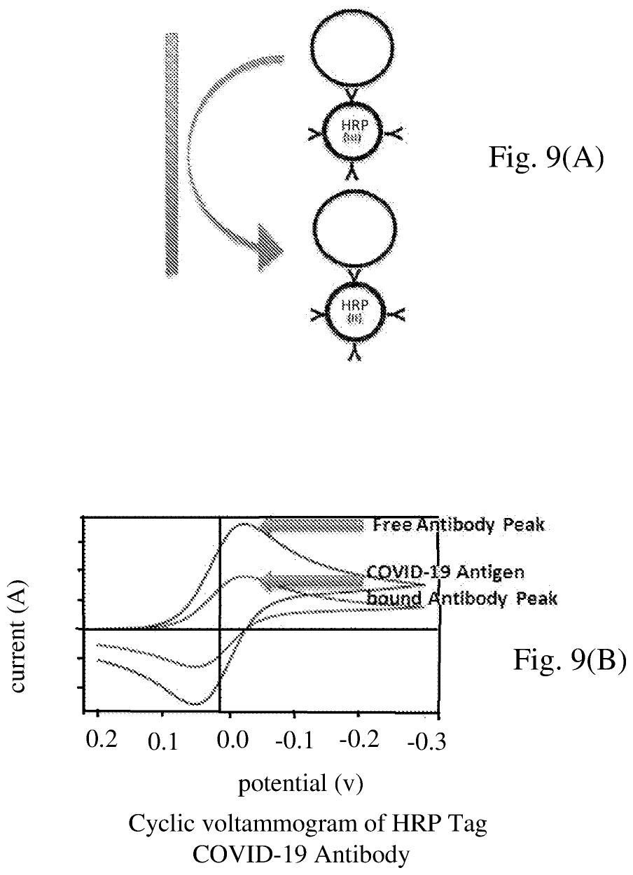

Once the antigen-antibody reaction is stabilized a step of measuring a peak value of redox current of the SARS-CoV2 antigen-specific and electrochemically active immunoreceptor is performed by implementing cyclic voltammetry (FIG. 9(A)-(B)), preferably by applying a specific voltage profile to the electrode arrangement as a function of time. A peak value of redox current is measured.

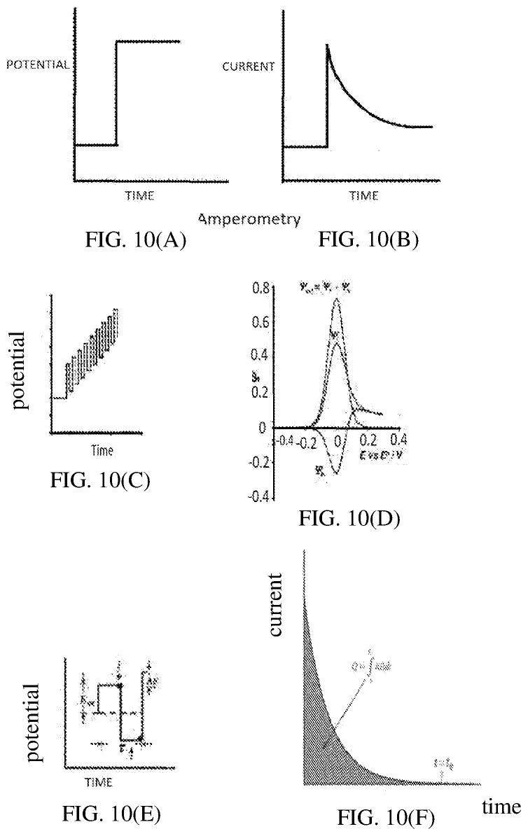

In the method of the present invention, the measurement of the peak value of redox current of the SARS-CoV2 antigen-specific and electrochemically active immunoreceptor, is also performed by a square wave voltammetry (SWV) or differential pulse voltammetry (DPV) as shown in FIG. 10.

Alternatively, a step of amperometry is performed (FIG. 10(A)-(F)) by applying a constant potential to the SARS-CoV2 antigen-specific and electrochemically active immunoreceptor and the resulting peak redox current is measured.

It is also within the purview of the invention to use Coulometry (FIG. 10(A)-(F)) to measure a peak value of redox current of the SARS-CoV2 antigen-specific and electrochemically active immunoreceptor, by applying a constant potential, chosen so that the bioanalyte reacts completely. As the electrolysis progresses the analyte's concentration decreases, as does the current. The resulting current-versus-time profile for controlled-potential coulometry is shown in (FIG. 10(F)). Integrating the area under the curve from t=0 to t=tₑ gives the total charge.

Finally, a concentration of SARS-CoV2 specific antigen in the biological sample is determined, by linearly matching the measured redox current with a corresponding reference redox current of the device in a database to retrieve and display the concentration of COVID-19 specific antigen in the biological sample. An exemplary database is shown as in the following Table.

TABLE

| SARS-CoV2 antigen | |
|---|---|
| concentration | SWV Peak current |
| 0.1 nano Molar | 10 μA |
| 0.3 nano Molar | 7 μA |
| 0.5 nano Molar | 3 μA |
| 1 nano Molar | 1.5 μA |
| 3 nano Molar | 1 μA |
| 5 nano Molar | 0.5 μA |

In the present invention, PCR thermal cycler can also be used to amplify SARS-CoV2 antigen DNA and then use the specific antibodies tagged or conjugated with electrochemical labels for electrochemical detection of SARS-CoV2 antigen.

The subject matter of the invention is now illustrated in the form of the following examples. These examples are provided for purpose of illustration only and shall not be construed as limiting the scope of the claimed invention.

### Example 1

### Cyclic Voltammetry Determination of SARS-CoV2 Specific Antigen in Biological Samples

A few micro litre volume of HRP Tag/conjugate SARS-CoV2 specific antibodies with suitable concentration (about 10 nano Molar), which can vary in nanomolar (nM) to millimolar (mM) is prepared in a phosphate buffer saline. The biological sample is added in Solution-A (Sol-A) (FIG. 8(a)) and a sufficient time (about 5 minutes) is provided to bind SARS-CoV2 antigen with HRP Tag SARS-CoV2-electrochemically active IgG monoclonal antibody, as shown in FIG. 8(a) and FIG. 8(b). The SARS-CoV2 antibody will bind with SARS-CoV2 antigen in the solution to form SARS-CoV2 Antigen-SARS-CoV2-Antibody, as shown in FIG. 8(b).

After inserting the printed electrode into the biosensor, which is configured in cyclic voltammetry techniques, the bound complex (Sol-C) is applied on the printed electrode. SARS-CoV2 antibody is tag/conjugated with HRP, where HRP is electrochemically active molecule with iron redox centre. HRP tagged/conjugates SARS-CoV2 antibody will give the cyclic voltammogram as shown in FIG. 9(B). After analysing the redox current change and redox peak shift, presence of SARS-CoV2 antigen is detected in the biological sample.

### Example 2

Detection and Measurement of SARS-CoV2 Antigen without an Electrode Reactivity Enhancement Agent

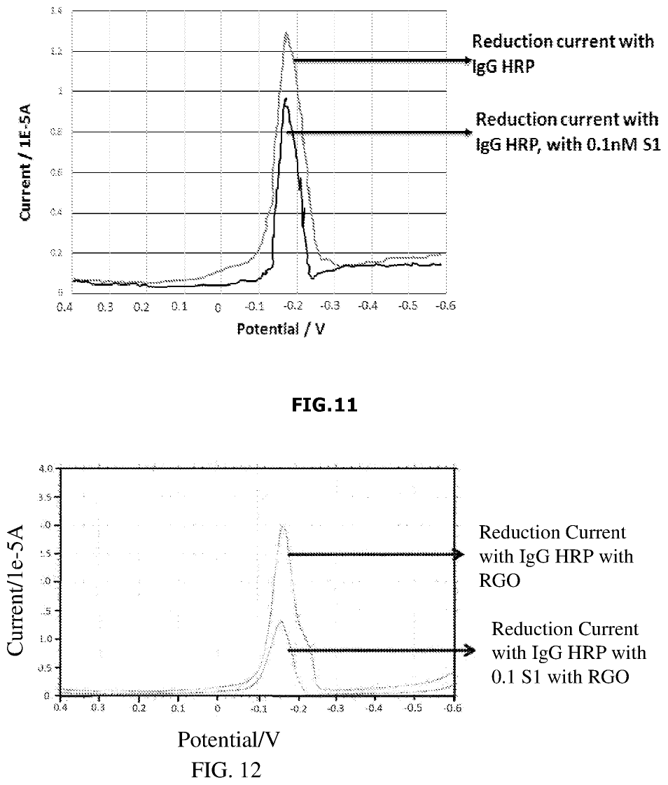

Spiked S1-Spike Protein (antigen) and SARS-CoV2 antigen specific IgG antibodies that are conjugated/tagged with horseradish peroxidase (immunoreceptor) are obtained from The Native Antigen Company, UK. Ten nanomolar (nM) concentration of the immunoreceptor is mixed with a combination of Stabilcoat (Sigma) and saline buffer. Six micro litre (μl) of this solution is dispensed on the carbon screen printed electrode of the device of the present invention and dried at room temperature. The SARS-CoV2 antigen with 0.1 nM concentration is spiked in viral transport media (VTM). Ten μl of this sample is mixed with 40 μl of saline buffer to create 50 μl biological sample volume. The sample is then dispensed on the electrochemical device functionalized with the immunoreceptor and the stabilization agent (50% Stabilcoat solution in saline buffer). After allowing a time of about 5 minutes for the antigen-antibody binding chemistry to reach an equilibrium, square wave voltammetry measurement is performed. As shown in FIG. 11, the peak current reduces indicating the presence of SARS-CoV2 antigen in the VTM solution. For reference, the current value obtained without antigen is also plotted in same figure.

### Example 3

Detection and Measurement of SARS-CoV2 Antigen with Electrode Reactivity Enhancement Agent:

Spiked S1-Spike Protein (antigen) and SARS-CoV2 antigen specific IgG antibodies that are conjugated/tagged with horseradish peroxidase (immunoreceptor) are obtained from The Native Antigen Company, UK. Reduced Graphene Oxide (RGO) powder is obtained from Sigma. Ten nano molar (nM) concentration of the immunoreceptor is mixed with a combination of Stabilcoat (Sigma, 50% solution in saline buffer), 0.001% Reduced graphene oxide solution in saline buffer. Six micro litres (pi) of this solution is dispensed on the carbon screen printed electrode of the device of the present invention and dried at room temperature. The antigen with 0.1 nM concentration is spiked in viral transport media (VTM). Ten μl of this sample is mixed with 40 μl of saline buffer to create 50 μl of biological sample volume. The sample is then dispensed on the electrochemical device functionalized with immunoreceptor, electrode reactivity enhancement agent and stabilization agent. After a waiting time of about 5 minutes for the antigen-antibody binding chemistry to reach equilibrium, square wave voltammetry measurement is performed. As shown in square wave voltammetry waveform of FIG. 12, the peak current reduces indicating the presence of SARS-CoV2 antigen in VTM solution. For reference, the current value obtained without antigen is also plotted in same figure. It is observed that the current values have increased very substantially (both without Antigen and with Antigen) compared to FIG. 11, due to the presence of electrode reactivity enhancement agent. In particular, without antigen, the peak current in FIG. 11 is 13 micro ampere (μA), and with 0.1 nM antigen it comes down to about 10 μA (change in current is 3 μA). On the other hand, with electrode reactivity enhancement agent as shown in FIG. 12, the respective currents are 30 μA and 13 μA (change in current is 17 μA.)

### Example 4

Quantitative Estimation of SARS-CoV2 Antigen without Electrode Reactivity Enhancement Agent

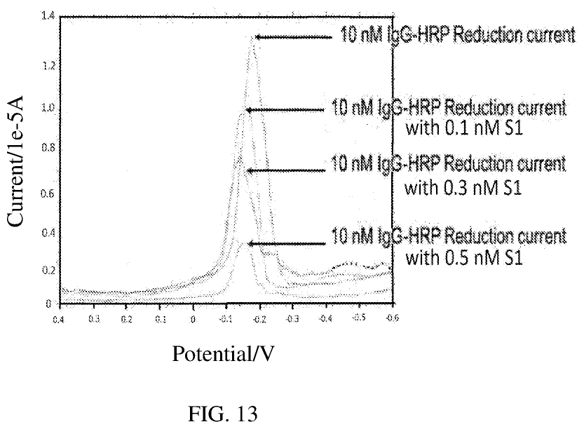

Spiked S1-Spike Protein (antigen) and SARS-CoV2 antigen specific IgG antibodies conjugated/tagged with horseradish peroxidase (immunoreceptor) are obtained from The Native Antigen Company, UK. Reduced graphene oxide (RGO) powder is obtained from Sigma. Ten nM concentration of immunoreceptor is mixed with a combination of Stabilcoat (Sigma, 50% solution in saline buffer), and saline buffer. Six μl of this solution is dispensed on the carbon screen printed electrode of the present invention and dried at room temperature. Several such electrochemical devices are prepared to perform measurements for different concentration of SARS-CoV2 antigen. The antigen with concentration ranging from 0.1 nM to 0.5 nM concentration is spiked in viral transport media (VTM), to prepare several solutions of test sample with varying SARS-CoV2 antigen concentration. Ten μl of each of sample is mixed with 40 μl of saline buffer to create 50 μl of the biological sample volume. The samples are then dispensed on different electrochemical devices functionalized with immunoreceptor, electrode reactivity enhancement agent and stabilization agent, as described earlier in this example. After allowing a time of about 5 minutes for the antigen-antibody binding chemistry to reach equilibrium, square wave voltammetry measurement is performed and the change in peak current from reference current is noted. As shown in FIG. 13, the change in peak current is a function of SARS-CoV2 antigen concentration. As the concentration increases, the change in current also increases and this information is used to quantitatively estimate SARS-CoV2 Antigen in the biological sample. The quantitative estimation of the SARS-CoV2 antigen in any arbitrary test sample is done by comparing the redox peak current value with the database Table, as shown earlier in Paragraph [0087].

### ADVANTAGES

The device and method of the present invention can provide qualitative or quantitative or both types of detection of SARS-CoV2 antigens, with immunoreceptor chemistry functionalized on the device.

The device and method of the present invention can also work in liquid phase without membrane functionalization, which can give more stability of sensing immune chemistries.

We claim:

1. A device 100 for collecting and retaining a biological sample, for measuring a concentration of a SARS-CoV2 specific antigen in a biological sample, comprising:
(i) at least a pair of conductive tracks 102a, 102b are disposed on a substrate 101;
(ii) at least a pair of electrodes 103a, 103b are connected to the at least pair of conductive tracks 102a, 102b; and
(iii) a SARS-CoV2 antigen-specific and electrochemically active immunoreceptor 105 that is conjugated with at least an electrochemically active substance, is configured to be in chemical contact with the at least pair of electrodes 103a, 103b and the biological sample.

2. The device 100 as claimed in claim 1, wherein the material for the substrate is a rigid material, preferably, ceramic, glass or a flexible material, preferably, polyvinylchloride (PVC), polyethylene terephthalate (PET), polymethylmethacrylate (PMMA), epoxy fiber composites, polyamides composites and paper.

3. The device as claimed in claim 1, wherein the at least pair of electrodes 103a, 103b include patterned electrodes.

4. The device 100 as claimed in claim 1, wherein a membrane 104 is disposed on the at least pair of electrodes 103a, 103b.

5. The device 100 as claimed in claim 1, wherein the SARS-CoV2 antigen-specific and electrochemically active immunoreceptor 105 is selected from monoclonal antibodies or polyclonal antibodies, preferably human immunoglobulin M(IgM) or human immunoglobulin G(IgG), or a combination thereof.

6. The device 100 as claimed in claim 1, wherein the electrochemically active substance is one of horseradish peroxidase (HRP), histidine, biotin, alkaline phosphatase or a combination of these substances, gold, silver or an alloy of these metals.

7. The device 100 as claimed in claim 1, wherein the SARS-CoV2 antigen-specific and electrochemically active immunoreceptor 105 includes at least an electrode reactivity enhancement agent, selected from reduced graphene oxide (rGO), carbon nanotubes (CNT), metal nano particles, metal oxide nano particles or a combination thereof.

8. The device 100 as claimed in claim 1, wherein the SARS-CoV2 antigen-specific and electrochemically active immunoreceptor 105 includes at least an antibody stabilization agent, selected from ELISA stabilization buffer, a plate stabilizer or a combination thereof.

9. The device 100 as claimed in claim 1, wherein the device 100 is disposed in a cartridge or a cassette.

10. A device holder 200 comprising:
(i) a device detection and signal conditioning means disposed in a housing 201;
(ii) a USB connector 202 disposed at one end of the housing 201 and a device insertion port 203 is disposed at the other end of the housing 201; and
(iii) the device 100 of claim 1 being configured to connect to the housing 201 through the device insertion port 203.

11. A point-of-care biosensor 300 for measuring a concentration of a SARS-CoV2 specific antigen in a biological sample, the biosensor comprising:
(i) a micro USB member 302, a micro SD card 303, a display member 304 and a device insertion port 305, are disposed in a housing 301;
(ii) the device 100 is disposed to connect to the housing 301 through the device insertion port 305,
the device 100 including
the at least pair of conductive tracks 102a, 102b that are disposed on a substrate 101,
the at least pair of electrodes 103a, 103b that are connected to the at least pair of conductive tracks 102a, 102b, and
the SARS-CoV2 antigen-specific and electrochemically active immunoreceptor 105 that is conjugated with the at least electrochemically active substance and is configured to be in chemical contact with the at least pair of electrodes 103a, 103b and the biological sample; and
(iii) a processing means 307 is disposed in the housing 301 and configured to measure a peak value of redox current of the SARS-CoV2 antigen-specific and electrochemically active immunoreceptor 105, from a redox potential applied to the device 100,
(iv) the processing means 307 is also configured to measure a concentration of SARS-CoV2 specific antigen in the biological sample, by linearly matching the measured redox current with a corresponding reference redox current of the device and retrieving the matched concentration of the SARS-CoV2 specific antigen; and
(v) the processing means 307 is further configured to display the measured concentration of the SARS-CoV2 specific antigen.

12. The point-of-care biosensor, as claimed in claim 11, wherein a database member 306 including standard values of SARS-CoV2 antigen concentrations along with corresponding redox currents, is connected to the processing means 307.

13. A method for measuring a concentration of SARS-CoV2 specific antigen, comprising the steps of:
(a) collecting a desired biological sample of reduced volume and diluting in a saline buffer or storing in viral transport media (VTM);
(b) contacting the biological sample with the SARS-CoV2 antigen-specific and electrochemically active immunoreceptor of the device of claim 1 and stabilizing the antigen-antibody reaction;
(c) measuring a peak value of redox current of the SARS-CoV2 antigen-specific and electrochemically active immunoreceptor of the device;
(d) determining a concentration of SARS-CoV2 specific antigen in the biological sample, by linearly matching the measured redox current with a corresponding reference redox current of the device and retrieving the matched concentration of the SARS-CoV2 specific antigen; and
(e) displaying the concentration of the SARS-CoV2 specific antigen.

14. The method as claimed in claim 13, wherein the biological sample is urine, blood, saliva, sweat, serum, or a nasopharyngeal culture.

15. The method as claimed in claim 13, wherein the volume of the biological sample is in the range of 1-50 microlitres (μL) and the saline buffer volume for dilution is in the range of 10-100 microlitres (μL).

16. The method as claimed in claim 13, wherein the SARS-CoV2 antigen-specific and electrochemically active immunoreceptor is selected from monoclonal antibodies or polyclonal antibodies, preferably human immunoglobulin M(IgM) or human immunoglobulin G(IgG), or a combination thereof.

17. The method as claimed in claim 13, wherein the electrochemically active substance is one of horseradish peroxidase (HRP), histidine, biotin, alkaline phosphatase or a combination of these substances, gold, silver or an alloy of these metals.

18. The method as claimed in claim 13, wherein the SARS-CoV2 antigen-specific and electrochemically active immunoreceptor includes the at least electrode reactivity enhancement agent, selected from reduced graphene oxide (rGO), carbon nanotubes (CNT), metal nano particles, metal oxide nano particles or a combination thereof.

19. The method as claimed in claim 13, wherein the SARS-CoV2 antigen-specific and electrochemically active immunoreceptor includes the at least antibody stabilization agent, selected from ELISA stabilization buffer, a plate stabilizer or a combination thereof.

20. The method as claimed in claim 13, wherein the measurement of the peak value of redox current of the SARS-CoV2 antigen-specific and electrochemically active immunoreceptor, is performed by square wave voltammetry (SWV), differential pulse voltammetry (DPV), amperometry or coulometry.
